(19) United States
(12) Patent Application Publication
(10) Pub. No.: US 2023/0390554 A1
(43) Pub. Date: Dec. 7, 2023

(54) BRAIN STIMULATION DEVICE WITH TARGETED INJECTABLE DRUG DELIVERY

(71) Applicant: Epic Neuro, Inc., Fountain Valley, CA (US)

(72) Inventors: James William PHILLIPS, Fountain Valley, CA (US); Robert M. ABRAMS, Los Gatos, CA (US)

(21) Appl. No.: 18/329,279

(22) Filed: Jun. 5, 2023

Related U.S. Application Data
(60) Provisional application No. 63/348,805, filed on Jun. 3, 2022.

(51) Int. Cl. A61N 1/32; A61N 1/05; A61N 1/36; A61M 39/02

(52) U.S. Cl. CPC A61N 1/327; A61N 1/0529; A61N 1/3605; A61M 39/0208; A61M 2210/0693; A61M 2039/0205

(57) ABSTRACT

Systems and methods are described, which provides electrical stimulation to a person, while also facilitating delivery of a drug through the system to a target region of the brain. The electrical stimulation to the target region of the brain increases a permeability of a blood brain barrier.

### PRIORITY CLAIM

This patent application claims priority to U.S. provisional patent application No. 63/348,805, titled “BRAIN STIMULATION DEVICE WITH TARGETED INJECTABLE DRUG DELIVERY” and filed on Jun. 3, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Electric brain stimulation has been shown to be a potentially effective treatment for a number of brain disorders, including epilepsy, migraine, fibromyalgia, major depression, stroke rehabilitation, and Parkinson's disease. While generally regarded as safe when following standard protocols, electric brain stimulation has been shown to increase the permeability of the blood brain barrier (BBB), allowing otherwise impermeable drugs to reach target areas. Brain endothelial cells, which form the endothelium of cerebral micro vessels, are responsible for the majority of resistance to substances. The integrity of the BBB is essential for the health and proper functioning of brain tissue. However, a temporary breakdown of the BBB and increased permeability may allow for improved advanced drug delivery methods. Electric stimulation may temporarily disrupt the BBB through either the paracellular or transcellular pathway. The type and extent of BBB disruption generally depends on the stimulation parameters, such as location, amplitude, polarity, duration, and frequency. Low frequency high amplitude pulses, applied for a short duration can electroporate endothelial cells, opening transcellular pathways. High frequency low amplitude stimulation applied for a longer duration may disrupt tight junctions, increasing the permeability of the BBB through the paracellular pathway.

Electric brain stimulation has been shown to be effective at treating a variety of Central Nervous System (CNS) disorders. For example, electrochemotherapy, tumor treating fields (TTFields), deep-brain stimulation (DBS), and irreversible electroporation have all shown clinical benefit. Electroporation is predominantly reversible at electric fields less than 400 V cm-1 in the brain and reversibly disrupts the BBB. By combining the benefits of electric brain stimulation with improved drug delivery to target brain regions, it is possible to achieve an additive benefit. A need exists for a method and system that allows targeted brain stimulation to achieve the benefits of neuromodulation, along with drug delivery to that location, and using the electric stimulation to increase permeability of the BBB in that region.

### SUMMARY

A device for electrical stimulation of a subject's brain is provided, the device comprising: a case comprising electronics configured to generate electrical pulses, the case including an opening that extends through the case; a probe coupled to the case and including a lumen in communication with the opening of the case, wherein the opening and the lumen are configured to receive a drug delivery device to facilitate drug delivery to a target region of the brain; at least one electrode disposed on the probe and configured to deliver electrical stimulation to the target region of the brain, wherein the electrical stimulation increases a permeability of a blood brain barrier in order to increase an effect of the drug on the target region of the brain.

In one aspect, the probe is flexible.

In another aspect, the lumen includes a seal or diaphragm to minimize drug backflow.

In one aspect, the drug delivery device comprises a syringe. In other aspects, the drug delivery device comprises a needle. In another aspect, the drug delivery device comprises a flexible tube.

In some aspects, the drug delivery device is implanted in the patient.

A system for electrical stimulation of a subject's brain is provided, the device comprising: a first case comprising first electronics, the first case including a first opening that extends through the first case; a first probe coupled to the first case and including a first lumen in communication with the first opening of the first case, wherein the first opening and the first lumen are configured to receive a first drug delivery device to facilitate drug delivery to a target region of the brain; at least one electrode disposed on the first probe; a second case comprising second electronics, the second case including a second opening that extends through the second case; a second probe coupled to the second case and including a second lumen in communication with the second opening of the second case; at least one electrode disposed on the second probe; wherein the first and second electronics are configured to generate pulses with opposite polarity such that electric current flows from the first probe through the target region to the second probe.

In one aspect, the electrical current is further configured to flow from the second probe under or through the a scalp to the first case to complete a current loop.

In another aspect, the second opening and the second lumen are configured to receive a second drug delivery device to facilitate drug delivery to the target region of the brain

In one aspect, the system includes a “T” or “Y” shaped coupler having a first branch configured to enter the first opening and a second branch configured to enter the second opening, allowing the drug to be administered through both the first and second lumens the first drug delivery device.

In one aspect, the drug delivery device is implanted in the patient.

A device for electrical stimulation of a subject's brain is provided, the device comprising: a remote pulse generator; a neurostimulator adapted to be implanted in a patient's brain, the neurostimulator being electrically coupled to the remote pulse generator, the neurostimulator comprising: a case including an opening that extends through the case; a probe coupled to the case and including a lumen in communication with the opening of the case, the probe being configured to be implanted in the subject's brain, wherein the opening and the lumen are configured to receive a drug delivery device to facilitate drug delivery to a target region of the brain; at least one electrode disposed on the probe and configured to deliver electrical stimulation to the target region of the brain, wherein the electrical stimulation increases a permeability of a blood brain barrier in order to increase an effect of the drug on the target region of the brain.

In one aspect, the lumen includes a seal or diaphragm to minimize drug backflow.

In another aspect, the remote pulse generator is implanted in the patient.

A device for electrical stimulation of a subject's brain is provided, the device comprising: a remote module comprising a pulse generator and a drug pump; a neurostimulator adapted to be implanted in a patient's brain, the neurostimulator being electrically and fluidly coupled to the remote pulse generator, the neurostimulator comprising: a case including an opening that extends through the case; a probe coupled to the case and including a lumen in communication with the opening of the case, the probe being configured to be implanted in the subject's brain, wherein the opening and the lumen are configured to receive a drug from the remote module to facilitate drug delivery to a target region of the brain; at least one electrode disposed on the probe and configured to deliver electrical stimulation to the target region of the brain, wherein the electrical stimulation increases a permeability of a blood brain barrier in order to increase an effect of the drug on the target region of the brain.

In one aspect, the remote module comprises a controller configured to turn drug delivery on and off.

In another aspect, the remote module comprises a controller configured to adjust a flow rate of the drug.

A system for electrical stimulation of a subject's brain is provided, the system comprising: an endovascular device configured to be fluidly coupled with a blood vessel of the subject, the endovascular device comprising a pulse generator and a drug pump configured to deliver a drug to a first target region in the subject's brain; a neurostimulator adapted to be implanted in a patient's brain, the neurostimulator comprising: a case including an opening that extends through the case; a probe coupled to the case and including a lumen in communication with the opening of the case, the probe being configured to be implanted in the subject's brain; at least one electrode disposed on the probe and configured to deliver electrical stimulation to a second target region of the brain, wherein the electrical stimulation increases a permeability of a blood brain barrier in order to increase an effect of the drug.

In some aspects, the neurostimulator is configured to sense EEG signals from the target region.

In one aspect, the first target region and the second target region are the same.

In another aspect, the first target region and the second target region are different.

A method of treating a target region of a brain of a patient is provided, comprising: implanting a neuro stimulator in the patient's brain such that a case of the neuro stimulator is beneath a scalp but above a skull of the patient and the probe is disposed in the brain with an electrode in or near the target region of the brain; inserting a drug delivery device through a hole in the case and into a lumen of the probe to deliver a drug to the target region of the brain; and delivering electrical stimulation to the target region of the brain with the electrode to increase a permeability of a blood brain barrier.

### BRIEF DESCRIPTION OF THE DRAWINGS

FIGS. 1A-1B show a neurostimulator device with a top view (101) and a side view (102).

FIG. 2 shows an example implementation of the device from FIGS. 1A-1B, where a brain (201) is surrounded in part by a skull (202), shown in the figure with hash lines.

FIG. 3 shows another example implementation involving two devices. In this example, a first device (301) is implanted so that the probe (302) is positioned so that the distal end of the probe is near one edge of a target region (303).

FIG. 4 shows another example implementation in which the electrical stimulation is produced by a pulse generator which is remote, but electrically connected to the probe electrodes.

FIG. 5 shows another example implementation in which electrical stimulation is produced by a pulse generator which is remote, and drug delivery is also provided by an implantable remote device.

FIG. 6 shows another example implementation in which the vascular system is used to direct the flexible tubing to a target location.

FIG. 7 shows an example system in which stimulation is delivered directly via probes inserted through a drill hole in the skull and endovascularly.

FIG. 8 shows an example system in which a direct stimulation device and an endovascular device stimulate separate target regions.

FIG. 9 shows two example coding systems, which allows information to be passed between implantable devices using variations in pulses delivered.

FIG. 10 shows another example of a coding system, in which a waveform of a pulse is shown (1001).

### DETAILED DESCRIPTION

While certain embodiments have been provided and described herein, it will be readily apparent to those skilled in the art that such embodiments are provided by way of example only. It should be understood that various alternatives to the embodiments described herein may be employed, and are part of the invention described herein.

Provided herein is a method and system whereby a target region in a brain of a person may be electrically stimulated, and that a drug or medication may be administered directly or indirectly to the target region, wherein the electrical stimulation increases a permeability of a BBB in order to increase an effect of the drug on the target region of the brain. The location, amplitude, waveform, and pulse frequency may be configured to optimize the permeability of the BBB. The increase in permeability may be permanent, but preferably the increase in permeability is transient, only lasting approximately as long as the drug or medication is present in the body of the person in sufficient quantity to have the desired effect on the brain, or while the concentration of the drug or medication at or near the target region in the brain is above a prespecified threshold, or while a measured effect of the drug or medication on the brain is above a prespecified threshold.

One example target region comprises a tumor. Another example target region comprises a brain feature, such as the Thalamus, Hippocampus, nucleus accumbens, prefrontal cortex, or some other identifiable component of the brain. Another example target region comprises a lesion that occurs due to a stroke. The target region and the disorder being treated may define which drug is optimal. The invention herein described is applicable regardless of which drug is used. Also, stimulation parameters may vary, depending on the drug or indication. For example, high frequency stimulation affects BBB differently than low frequency stimulation, and this effect may determine the optimal treatment.

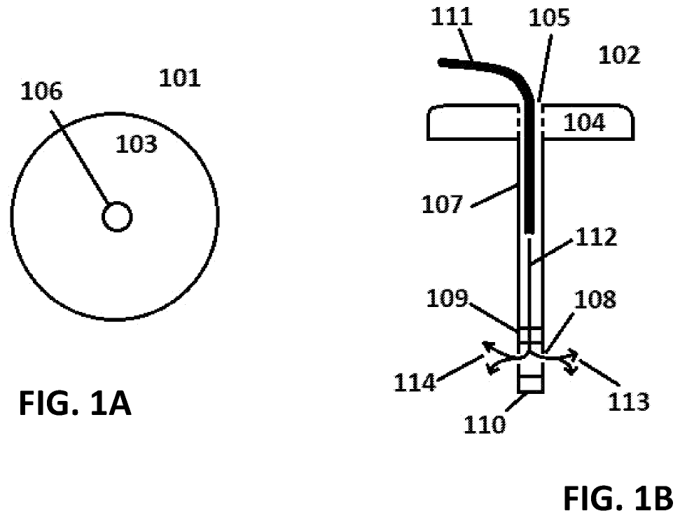

Turning to FIGS. 1A-1B, a device is shown with a top view (101) and a side view (102). The device comprises a case (103, 104), which encloses electronics which may be used to generate electric pulses. The electronics may comprise a battery, a processor with memory, and a pulse generator. The case may also comprise a coil, which is used for wireless power transfer, in which a coil external to the body generates an alternating magnetic field which is used to power the device. A hole (105, 106) may exist in the case. The device also may comprise a hollow probe (107), which has a certain length, where the length is dependent upon the location of the target region for stimulation and drug delivery. At least one hole may exist the end of the hollow probe (108). The hollow probe may be rigid. Preferably, the hollow probe is flexible, allowing the probe to be directed toward the target region. Near the end of the probe are two electrodes, one proximal (109) and one at the distal end of the probe (110). Wires (not labeled) may run down the sides of the probe, either interior or exterior, in order to administer electrical pulses between the two electrodes, generating an electric field in the region of the probe tip. At least one flexible tube or needle (111) is inserted into the hole in the case and fed into the hollow probe. The flexible tube or needle may be used to administer a drug (112), which travels through the remaining distance of the probe and through the hole near the end of the probe so that the drug flows (113, 114) from the probe into or near the target region of the brain. A seal or diaphragm may exist in the probe to minimize any of the drug flowing back through the probe and out through the hole in the case.

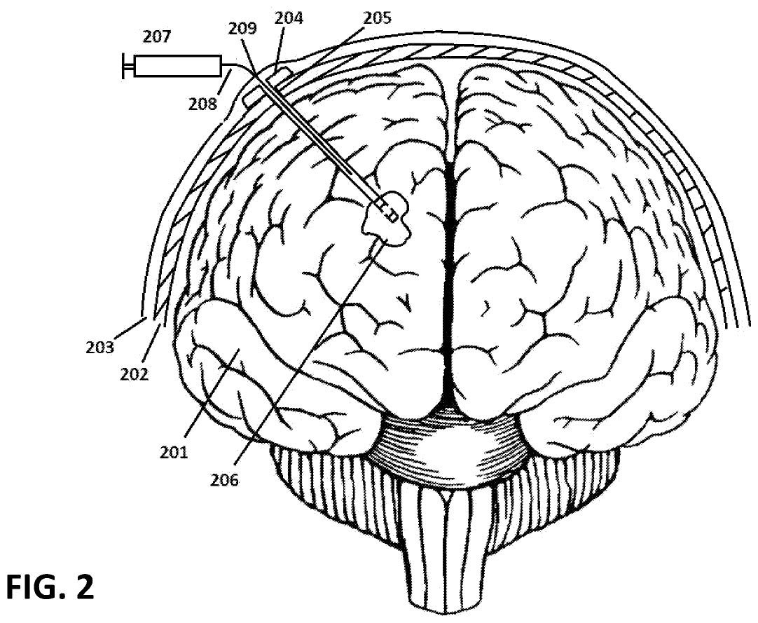

FIG. 2 shows an example implementation of the device from FIGS. 1A-1B, where a brain (201) is surrounded in part by a skull (202), shown in the figure with hash lines. The skull is covered by a scalp (203). The device is implanted beneath the scalp, where the scalp covers the case (204). The probe (107) is inserted into a small drill-hole in the skull (205), with the probe directed to the site of a target region (206), so that the electrodes (109, 110) stimulate in or around the target region, and the holes in the probe (108) are positioned to administer the drug in or near the target region. A syringe or some other injection mechanism (207) is positioned outside the head of the person, with a needle or catheter (208) piercing the skin at an injection location (209), and is inserted into the hole in the case (105) and into the hollow probe. The syringe may inject a drug (112) during stimulation, which exits the holes in the device (108) and is administered directly to the target region (206).

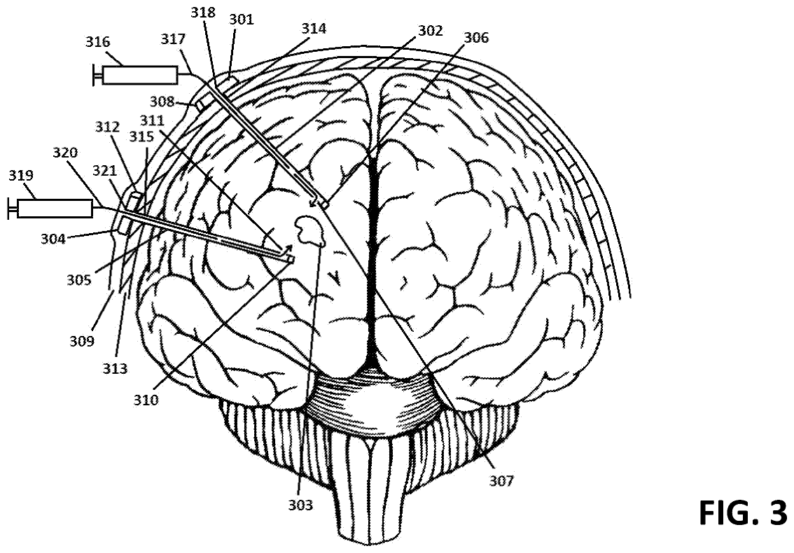

FIG. 3 shows another example implementation involving two devices. In this example, a first device (301) is implanted so that the probe (302) is positioned so that the distal end of the probe is near one edge of a target region (303). A second device (304) is implanted at a different location, with a probe (305) which is positioned so that the distal end of the probe is near the opposite side of the target region from the distal end of the probe of the first device. The first device comprises a probe electrode (306) at the distal end of the probe and a hole (307) in the probe tube near the probe electrode. The first device also comprises a case electrode (308), which is on the exterior of the case under the scalp (309). The second device also comprises a probe electrode (310) at the distal end of the probe and a hole (311) in the probe tube near the probe electrode, as well as a case electrode (312). The first device and second device may comprise pulse generators, which are timed to generate pulses at or near the same time with opposite polarity, such that the electric current flows from the probe electrode of the first device, through the target region, to the probe electrode of the second device. A current return path may proceed from the case electrode of the second device, under or through the scalp, to the case electrode of the first device, thereby completing a current loop. In this configuration, the skull (313) acts as a high impedance element to minimize any electric current shunting between the probe electrode and case electrode of the first device or between the probe electrode and case electrode of the second device. In this case, it may be preferable for the devices to further comprise a seal, which fills the space between the probe and the drill hole (314, 315), in order to prevent fluid ingress or egress through the drill hole, and thereby to prevent or minimize electric current flow through the drill hole. In addition, it may be necessary for the devices to comprise a seal or diaphragm in the probe tube or at the hole in the case, where the seal or diaphragm prevents fluid ingress or egress between the brain and the scalp which thereby minimizes electric current flow through the probe tubes, preventing or minimizing current shunting between the probe electrode and case electrode of the first device or between the probe electrode and case electrode of the second device. This seal or diaphragm may be able to be pierced by a needle or catheter to allow injection of a drug, and when the needle or catheter is extracted, the seal or diaphragm may close again to further prevent ingress or egress of the drug or other fluids between the brain and scalp.

A first syringe (316) may inject a drug through a needle or catheter (317), which pierces the scalp and enters the hole in the first device (318) and proceeds through the probe tube. The drug exits the needle or catheter, proceeding the rest of the way through the tube, and exiting a hole in the tube which is positioned, on, or near the target region. A second syringe (319) may also be used to inject a drug through a needle or catheter (320), which enters the hole in the second device (321) and proceeds through the probe tube. The drug exits the needle or catheter, proceeding the rest of the way through the tube, and exiting a hole in, on, or near the target region.

Alternately, a single syringe may inject a drug, and the needle or catheter may comprise a “T” or “Y” shaped coupler having a plurality of branches, where each branch enters the hole/lumen in the case of each of the devices, allowing the drug to be administered through both probe tubes via a single syringe.

It may be preferable for the first device or second device to not comprise a pulse generator, and to simply provide an electric connection between the interior and exterior of the skull. In this case, the current pulses may only be generated by one of the two devices, and the other device would act only as a conductive path through the skull, allowing the current loop. Alternately, the first device or second device may not comprise a probe electrode or a case electrode, and may use fluid inside the hollow probe to act as a conductive path for current pulses generated by the other device. In this case, the non-stimulating device may not comprise a diaphragm or seal inside the tube.

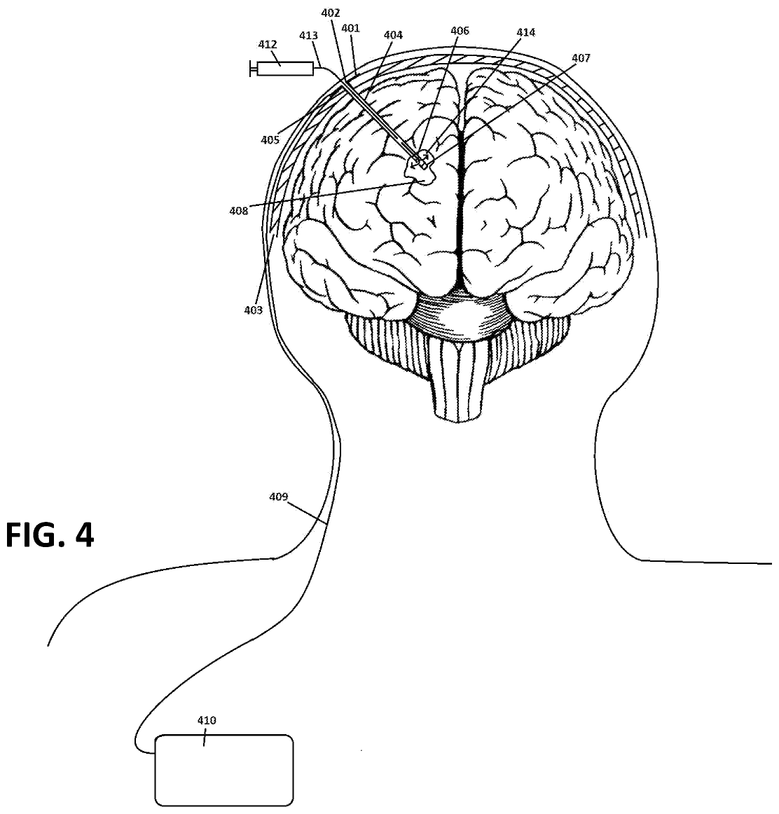

FIG. 4 shows another example implementation in which the electrical stimulation is produced by a pulse generator which is remote, but electrically connected to the probe electrodes. The pulse generator can be positioned external to a patient. Alternatively, the pulse generator can be implanted in a location outside of the patient's brain (e.g., in a patient's chest, arms, back, etc.). The device comprises a thin case or a flange (401), which comprises a hole or opening (402) and rests on the outside of the skull (403). A hollow probe (404) is affixed to the flange, and is inserted into a hole (405) in the skull, and positioned so that the two electrodes (406, 407) are in or near a target region in the brain (408). The electrodes are electrically connected through the thin case or flange, via a lead (409) to a remote pulse generator (410). The pulse generator administers electrical stimulation via the lead such that electric current flows between the two electrodes, thereby increasing the permeability of the region to a drug. A syringe (412) injects a drug through a needle or catheter (413), which pierces the scalp and enters the hole in the flange. The drug exits the needle or catheter, proceeding the rest of the way through the tube, and exiting (414) one or more holes in the tube which are positioned in, on, or near the target region. The probe or hole in the flange may comprise a diaphragm, which allows a needle catheter to penetrate to administer the drug, but does not otherwise allow significant fluid ingress or egress between the inside and outside of the skull.

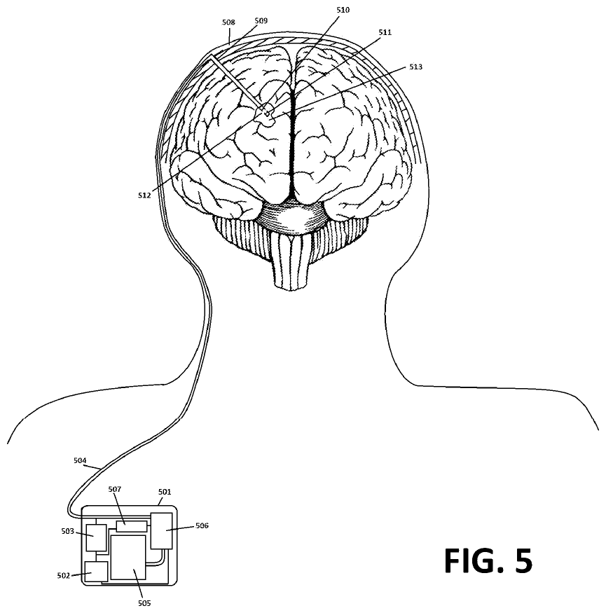

FIG. 5 shows another example implementation in which electrical stimulation is produced by a pulse generator which is remote, and drug delivery is also provided by an implantable remote device. In this example, the pulse generator and drug delivery system are both part of the same module. The remote module (501) comprises a battery (502) and pulse generator (503). The pulse generator is electrically connected to wires that run the length of a flexible tubing (504). The remote module also comprises a reservoir containing a drug (505) and a pump (506) or other means by which the drug may be delivered through the flexible tubing. The remote module may also comprise a controller (507), which turns drug delivery on and off, or adjusts the flow rate, and may also control stimulation. The flexible tubing may run underneath the skin and scalp (508), and through a drill-hole or craniotomy (509). The tube may comprise two electrodes (510, 511) and one or more holes near the distal end of the probe (512). The pulse generator may administer electrical stimulation between the two electrodes before, during, or after the pump or other means injects a drug through the tubing, which exits through the holes in, on, or near the target region (513).

In another aspect, the remote module may not comprise a reservoir and pump for the drug, and instead the drug may be injected using a syringe or catheter which goes through the skin and injects the drug directly into the flexible tubing. In an alternate aspect, the reservoir may be refilled with a drug using a needle or catheter which is fluidically coupled to the reservoir.

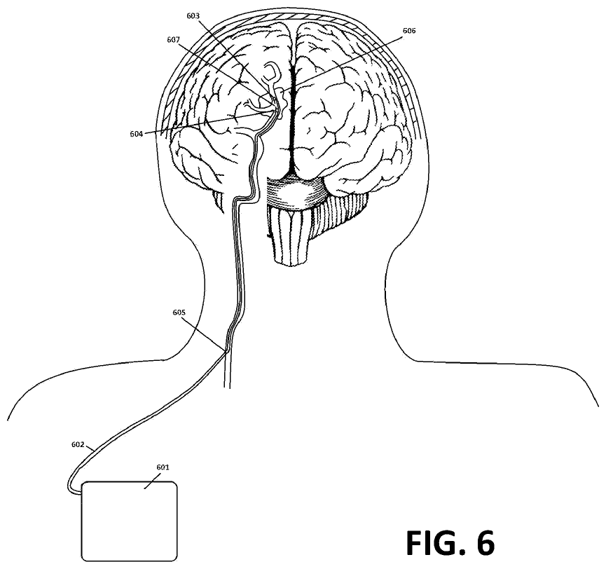

FIG. 6 shows another example implementation in which the vascular system is used to direct the flexible tubing to a target location. In this example, a remote device (601) comprises a pulse generator, a reservoir, and pump or other means to inject a drug into the flexible tubing (602). Wires from the pulse generator run in, on, or alongside the flexible tubing, and are electrically connected to two electrodes (603, 604). The tubing enters the vascular system via an entry point (605) and is positioned in a blood vessel in or near the target region (606). The tubing comprises a hole (607) near the distal end in order to allow the drug to enter the vascular system in or near the target region. The pulse generator may administer electric current flowing between the two electrodes in order to increase the permeability of the blood brain barrier in order to allow the drug injected in or near the target region to have an increased effect on the brain.

The electrodes may be incorporated into a stent, which lies on or near the inner surface of a blood vessel in or near the target region. The stent may allow for better securing of the electrodes in place, and may prevent or minimize drift. The drug may be injected using small doses or a low flow rate in order to allow for a greater effect on the target region.

It is not essential that the drug be administered directly to the target region. It may be beneficial to use a separate catheter or syringe to inject the drug into another region of the body, and allow normal circulation to bring the drug to the target region. However, by administering the drug directly to an area in or near the target region, the drug may be in a higher concentration in that area, avoiding the natural dilution of the drug by the circulatory system, thereby increasing the effect and potentially lessening the required dosage, resulting in fewer negative side effects from the drug.

In another aspect, the remote module may not comprise a reservoir and pump for the drug, and instead the drug may be injected using a syringe or catheter which goes through the skin and injects the drug directly into the flexible tubing. In an alternate aspect, the reservoir may be refilled with a drug using a needle or catheter which is fluidically coupled to the reservoir.

In the aforementioned examples, the electrodes were intended to provide stimulation. However, the device may further comprise an electroencephalograph (EEG) amplifier, wherein an EEG recording may be obtained through the electrodes. The device may further comprise a processor, memory, and a means to communicate with an external device. The EEG recording may be streamed or uploaded to the external device for further analysis or potential brain-machine interface (BMI).

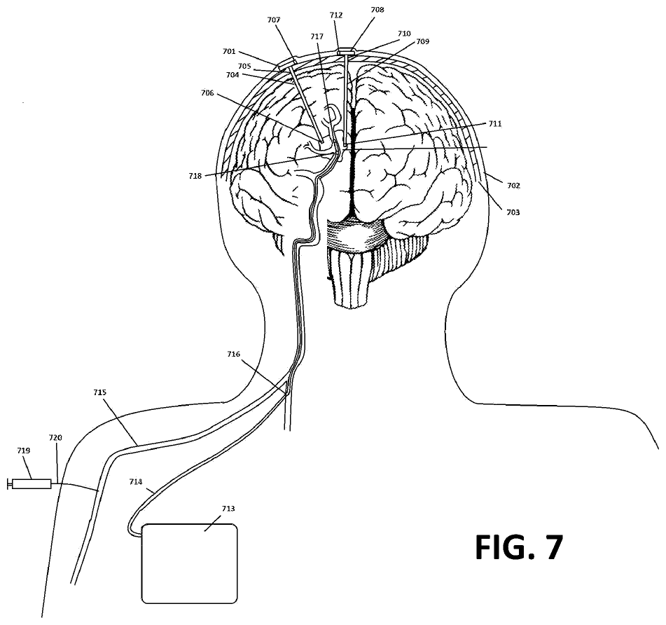

The example system in FIG. 6 may be combined with a system as shown in FIGS. 1A-1B, which allows the drug to be administered to the target region both via the vascular system and directly to brain tissue, thereby producing a potential additive effect. FIG. 7 shows an example system in which stimulation is delivered directly via probes inserted through a drill hole in the skull and endovascularly. A first direct stimulation device (701) is implanted beneath the scalp (702), where the case rests on the surface of the skull (703). A probe (704) is inserted through a drill hole (705), with an electrode (706) at the distal end of the probe. A second electrode (707) is on the side of the case. A second direct stimulation device (708) is implanted at a second location such that the case is underneath the scalp and rests on the surface of the skull. A probe (709) is inserted through a drill hole (710), with an electrode (711) at the distal end of the probe. A second electrode (712) is on the side of the case. The electrodes (706, 711) are positioned so that electric current flows through the target region, and the two case electrodes (707, 712) allow a return path for current underneath or through the scalp. The skull is high impedance and restricts current flow shunting between the probe electrode and case electrode of the same device. In general, the devices may further comprise a seal which fills the gap between the probe and the inner surface of each drill hole, preventing electric current from shunting through the drill-hole.

The endovascular device comprises a pulse generator (713), with a lead (714), which enters a blood vessel (715) at a prespecified location (716). The lead proceeds via the vasculature to a location at or near the target region. The device comprises two electrodes (717, 718) at or near the distal end of the lead. This device may generate stimulation pulses to affect the brain in the target region.

It may be that stimulation by itself may suffice to bring about the desired effect. In one example, the target region is the nucleus accumbens, and stimulation may be intended to treat substance abuse. In another example, the target region is the anterior nucleus of the thalamus, and stimulation may be intended to reduce seizures.

If the stimulation is intended to increase the permeability of the BBB, a syringe (719) may inject a drug through a needle or catheter (720) into a blood vessel in the person. The amount of drug that reaches the target region in this case may be diluted, but the increased permeability in the BBB may significantly improve the efficacy of the drug in or near the target region.

Both the direct stimulation devices and the endovascular device may sense EEG signals from around the target region. The EEG signals would be produced by recording the voltage potential between the two probe electrodes (706, 711), and/or by recording the voltage potential between the two lead electrodes (717, 718). This EEG recording may be used to estimate brain health, brain activity, changes in metabolism, or other biological measurements. The EEG recordings may also be used as part of a Brain-Machine-Interface (BMI). If one or more devices record EEG, the system may additionally comprise an EEG amplifier and an analog-to-digital converter (ADC). The EEG may be stored in memory in the endovascular pulse generator or the direct stimulation generator, or both.

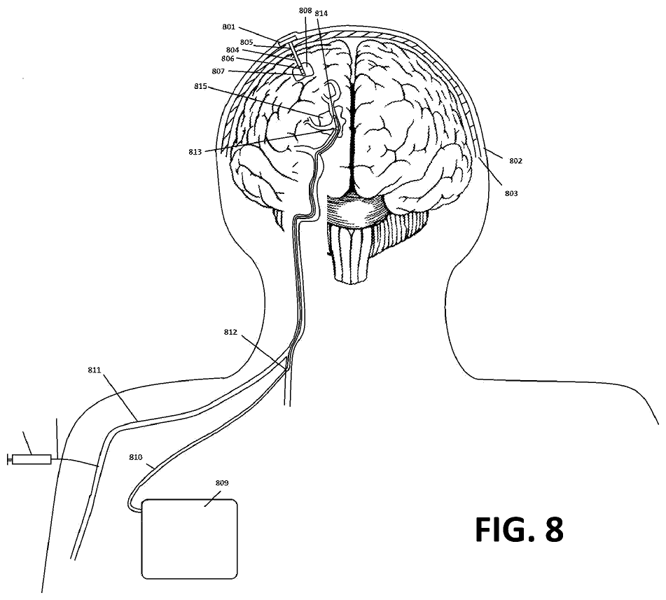

FIG. 8 shows an example system in which a direct stimulation device and an endovascular device stimulate separate target regions. A direct stimulation device (801) is implanted beneath the scalp (802) with the case resting on the outer surface of the skull (803). A probe (804) is inserted through a drill-hole (805) in the skull, and the probe is positioned so that the electrodes (806, 807) generate electrical stimulation between them that flows in or near a first target region (808). An endovascular device is shown with a pulse generator (809) implanted in the body with a lead (810) that enters a blood vessel (811) at a prespecified location (812). The lead is directed in the vasculature so that the two electrodes (813, 814) are positioned so that the electrodes generate electrical stimulation between them that flows in or near a second target region (815).

It may be that stimulation of multiple target regions by itself may suffice to bring about the desired effect. In one example, the target region is the nucleus accumbens, and stimulation may be intended to treat substance abuse. In another example, the target region is the anterior nucleus of the thalamus, and stimulation may be intended to reduce seizures.

If the stimulation is intended to increase the permeability of the BBB, a syringe (719) may inject a drug through a needle or catheter (720) into a blood vessel in the person. The amount of drug that reaches the target region in this case may be diluted, but the increased permeability in the BBB may significantly improve the efficacy of the drug in or near the target region. Alternately, the direct stimulation device may comprise a hollow tube with a hole near the distal end so that the drug may be injected directly into the target region. Alternatively, the endovascular stimulation device may comprise a flexible tubing with a hole that allows the drug to be delivered into the bloodstream in or around the second target region.

It may be necessary for the endovascular pulse generator and one or both of the direct stimulation devices to communicate, or for two direct stimulation devices to communicate with each other. For example, it may be advantageous to synchronize pulses so that pulses are delivered at the same time or with a pre-defined offset. In another example, devices may provide battery end-of-life estimates, so that stimulation power may be adjusted to maximize usable life of the devices. The devices could use a wireless transmitter and receiver to communicate. However, since the devices are preferably battery powered, conservation of power is very important. In one aspect the implantable devices may communicate using stimulation pulses to encode data. The system shown in FIGS. 1A-1B may be referred to as the first device and the system shown in FIG. 6 may be referred to as the second device. In one aspect, the a first device is able to communicate with the a second device by generating pulses which are encoded, so that the second device may record the pulses using the EEG amplifier. Both devices may comprise a processor and memory as well as a pulse generator and EEG recorder, to allow communication between them using encoded pulses. For example, devices could use a form of communication similar to Morse Code, in which long pulses correspond to dashes and short pulses correspond to dots. Alternately, long pulses may represent high bits and short pulses may represent low bits, and communication is generated via a bit stream. This encoding may be separate from stimulation, keeping the pulse amplitude just high enough to allow communication, but not significantly affecting the permeability of the BBB, or the encoding may be part of the stimulation pulses, allowing stimulation and communication at the same time. If encoding is part of stimulation pulses, the pulse width may be varied. For example, short pulses may represent low bits (‘0’) and long pulses may represent high bits (‘1’). Alternatively, the time between pulses may be varied in order to encode data. For example, a short time between two pulses may represent low bits, and a long time between two pulses may represent high bits. Alternately, a higher frequency bit stream may be encoded as part of a single pulse. For example, if a pulse has a pulse width of 250 usec, the first 10 usec may be used to generate a bit stream (high-low pattern) which encodes data.

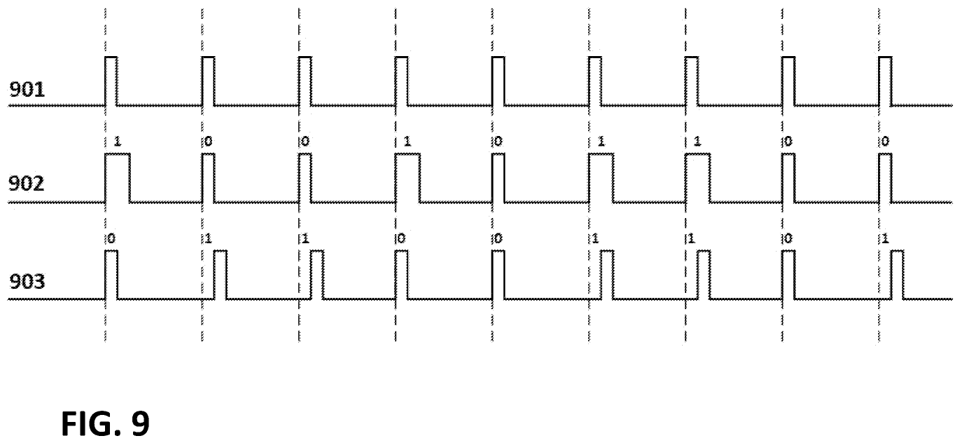

FIG. 9 shows two example coding systems, which allows information to be passed between implantable devices using variations in pulses delivered. The first pulse waveform (901) shows a standard pulse train, in which no data is encoded. The second pulse waveform (902) shows a pulse train in which pulse width encodes data. A wide pulse signifies a high bit, and a short pulse signifies a low bit. The third waveform (903) shows a pulse train in which pulses are shifted to encode data. This would require a pulse with a known ‘0’ value to allow the following pulses to be interpreted correctly. In this waveform, a pulse which is shifted signifies a high bit, and a pulse that is in phase with the starting pulse is a low bit. The data rate for this type of communication is very low, averaging one bit per pulse. However, the information may be quite small which is passed and may not be time critical.

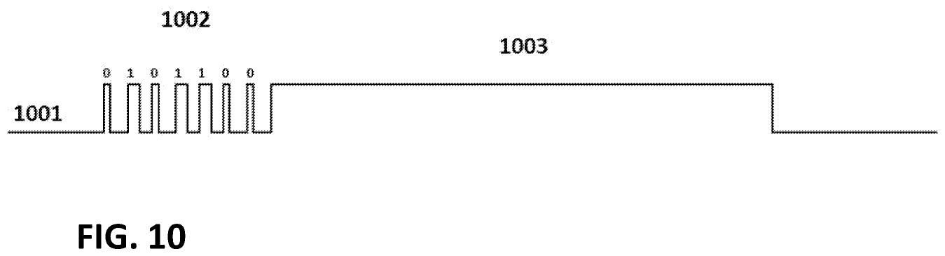

FIG. 10 shows another example of a coding system, in which a waveform of a pulse is shown (1001). At the start of the pulse, a pulse-width encoded message is sent (1002), followed by the remainder of the pulse. This may allow for a higher data rate. However, capacitance in the system may result in data loss, from very short pulses merging together

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A device for electrical stimulation of a subject's brain, the device comprising:
a case comprising electronics configured to generate electrical pulses, the case including an opening that extends through the case;
a probe coupled to the case and including a lumen in communication with the opening of the case, wherein the opening and the lumen are configured to receive a drug delivery device to facilitate drug delivery to a target region of the brain;
at least one electrode disposed on the probe and configured to deliver electrical stimulation to the target region of the brain, wherein the electrical stimulation increases a permeability of a blood brain barrier in order to increase an effect of the drug on the target region of the brain.

2. The device of claim 1, wherein the probe is flexible.

3. The device of claim 1, wherein the lumen includes a seal or diaphragm to minimize drug backflow.

4. The device of claim 1, wherein the drug delivery device comprises a syringe.

5. The device of claim 1, wherein the drug delivery device comprises a needle.

6. The device of claim 1, wherein the drug delivery device comprises a flexible tube.

7. The device of claim 1, wherein the drug delivery device is implanted in the patient.

8. A system for electrical stimulation of a subject's brain, the device comprising:
a first case comprising first electronics, the first case including a first opening that extends through the first case;
a first probe coupled to the first case and including a first lumen in communication with the first opening of the first case, wherein the first opening and the first lumen are configured to receive a first drug delivery device to facilitate drug delivery to a target region of the brain;
at least one electrode disposed on the first probe;
a second case comprising second electronics, the second case including a second opening that extends through the second case;
a second probe coupled to the second case and including a second lumen in communication with the second opening of the second case;
at least one electrode disposed on the second probe;
wherein the first and second electronics are configured to generate pulses with opposite polarity such that electric current flows from the first probe through the target region to the second probe.

9. The system of claim 8, wherein the electrical current is further configured to flow from the second probe under or through the scalp to the first case to complete a current loop.

10. The system of claim 8, wherein the second opening and the second lumen are configured to receive a second drug delivery device to facilitate drug delivery to the target region of the brain

11. The system of claim 8, further comprising a “T” or “Y” shaped coupler having a first branch configured to enter the first opening and a second branch configured to enter the second opening, allowing the drug to be administered through both the first and second lumens the first drug delivery device.

12. The system of claim 8, wherein the drug delivery device is implanted in the patient.

13. A device for electrical stimulation of a subject's brain, the device comprising:
a remote pulse generator;
a neurostimulator adapted to be implanted in a patient's brain, the neurostimulator being electrically coupled to the remote pulse generator, the neurostimulator comprising:
a case including an opening that extends through the case;
a probe coupled to the case and including a lumen in communication with the opening of the case, the probe being configured to be implanted in the subject's brain, wherein the opening and the lumen are configured to receive a drug delivery device to facilitate drug delivery to a target region of the brain;
at least one electrode disposed on the probe and configured to deliver electrical stimulation to the target region of the brain, wherein the electrical stimulation increases a permeability of a blood brain barrier in order to increase an effect of the drug on the target region of the brain.

14. The device of claim 13, wherein the lumen includes a seal or diaphragm to minimize drug backflow.

16. The device of claim 13, wherein the remote pulse generator is implanted in the patient.
